(19) United States
(12) Patent Application Publication
(10) Pub. No.: US 2020/0232039 A1
(43) Pub. Date: Jul. 23, 2020

(54) ADM2 GENE MARKER FOR DIAGNOSIS OR PROGNOSIS PREDICTION OF THYROID CANCER AND USES THEREOF

(71) Applicant: The Industry & Academic Cooperation in Chungnam National University (IAC), Daejeon (KR)

(72) Inventors: Minho Shong, Daejeon (KR); Yea Eun Kang, Daejeon (KR); Jeong Ho Lee, Daejeon (KR); Hyun Yong Koh, Daejeon (KR); Hyun Jung Kim, Daejeon (KR)

(21) Appl. No.: 16/445,322

(22) Filed: Jun. 19, 2019

(30) Foreign Application Priority Data
Jan. 22, 2019 (KR) 10-2019-0007842

(51) Int. Cl. C12Q 1/6886; G01N 33/574

(52) U.S. Cl. CPC C12Q 1/6886; G01N 33/57407; C12Q 2600/106; C12Q 2600/158; G01N 2333/5753; C12Q 2600/118

(57) ABSTRACT

The present invention relates to a composition for diagnosis or prognosis prediction of thyroid cancer, which includes an agent for measuring the expression level of mRNA of ADM2 gene or a protein thereof, a kit for diagnosis or prognosis prediction of thyroid cancer, which includes the composition, and a method for providing information for diagnosis or prognosis prediction of thyroid cancer using the composition or the kit.

### CROSS-REFERENCE TO RELATED APPLICATIONS

This application claims priority to and the benefit of Korean Patent Application No. 10-2019-0007842 filed in the Korean Intellectual Property Office on Jan. 22, 2019, the entire contents of which are incorporated herein by reference.

### TECHNICAL FIELD

The present invention relates to ADM2 (adrenomedullin 2) gene marker for diagnosis or prognosis prediction of thyroid cancer and uses thereof.

### BACKGROUND ART

The thyroid is a butterfly-shaped organ located in the lower portion of the thyroid cartilage and located in front of the airway, which is the air passage when breathing and functions to produce and store the thyroid hormone and to send it to the organ that needs it.

Thyroid cancer is a generic term for cancer which occurs in the thyroid, which is the most common malignancy of the endocrine system. Thyroid cancer is generally divided into “well-differentiated thyroid cancer” and “other thyroid cancer,” which is classified into as papillary carcinoma, follicular carcinoma, medullary thyroid carcinoma, and anaplastic carcinoma (undifferentiated thyroid cancer) according to the histological shape, the origin cells of cancer and the differentiation degree of the cancer. Among these, differentiated tumors such as thyroid papillary carcinoma and thyroid follicular carcinoma have a good prognosis in general but their survival rate is rapidly lowered when invading into surrounding tissues or metastasizing to other organs. Anaplastic carcinoma is a rare but notorious undifferentiated cancer in which most patients may die within six months.

Most of the thyroid cancer can be cured by the first surgery. Referring to disease stage, 80% to 85% of patients by disease stage are at low risk of death. However, some of them have a high-risk factor for recurrence. In fact, it has been reported that 20% of patients recurred and at most 50 to 60% of patients may die. Since it is more difficult to determine the prognosis after a recurrence of thyroid cancer than in the first operation, there are many studies on methods to reduce recurrence by analyzing factors related to the thyroid cancer's recurrence, and the recurrence frequency, recurrence site, disease-free period, recurrence treatment, treatment outcome and the like.

Meanwhile, Korean Patent No. 1587635 discloses a method for detecting methylation of a thyroid cancer-specific methylation marker gene for diagnosis of thyroid cancer, and Korean Patent No. 1845590 discloses a composition for predicting the prognosis of lung cancer using a gene. However, the ADM2 gene marker for diagnosis or prognosis prediction of thyroid cancer of the present invention and use thereof has not been described.

### SUMMARY OF THE INVENTION

The present invention has been made by the above demands, and the present inventors have performed RNA sequence analysis on a control group without cancer, an animal model of thyroid cancer under normal chow diet, and an animal model of thyroid cancer under high-fat diet in order to confirm the mechanism of clinical relevance of obesity and thyroid cancer. As a result of the analysis, it was confirmed that the expression level of ADM2 (adrenomedullin 2) gene was significantly different in the above three groups. In particular, the expression level of ADM2 gene in the animal model was increased in proportion to the size of thyroid cancer. Clinical sample analysis indicated that the expression of ADM2 protein in the obese group with thyroid cancer was increased compared to the normal weight group with thyroid cancer, and the expression of ADM2 protein in the obese group with recurrent thyroid cancer was significantly increased. The present inventors confirmed the possibility of the ADM2 gene or its protein as a biomarker for diagnosis or prognosis prediction of thyroid cancer, thereby completing the present invention.

In order to achieve the objects, the present invention provides a composition for diagnosis or prognosis prediction of thyroid cancer, the composition including an agent for measuring the expression level of mRNA of ADM2 (adrenomedullin 2) gene or its protein.

The present invention provides a kit for the diagnosis or prognosis prediction of thyroid cancer, the kit including the composition.

The present invention provides a method for providing information for the diagnosis of thyroid cancer by measuring the expression level of mRNA of the ADM2 gene or protein expressed from the gene from a biological sample isolated from a suspected thyroid cancer patient.

The present invention provides a method for providing information for prognosis prediction of thyroid cancer by measuring the expression level of mRNA of the ADM2 gene or protein expressed from the gene from a biological sample isolated from obese or recurrent patients with thyroid cancer.

The present invention provides a method of screening a therapeutic agent for thyroid cancer, the method including measuring the expression level of ADM2 gene or protein encoded by the gene after administering the candidate substance expected to be capable of curing the thyroid cancer.

The ADM2 gene of the present invention or protein thereof is a novel biomarker for the diagnosis, prognosis, or recurrence prediction of thyroid cancer, which can be useful for diagnosing thyroid cancer or predicting the severity of the disease and can be useful for screening therapeutic agents for thyroid cancer through analysis of expression level changes of the ADM2 gene or protein thereof.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A, 1B, 1C and 1D illustrate the results of RNA sequence analysis of gene expression levels in tissues of a control group without cancer (control), an animal model of thyroid cancer under normal chow diet (NCD), and an animal model of thyroid cancer under high-fat diet (HFD).

FIG. 2 illustrates the results of confirming the expression of AMD2 protein in NCD and HFD by immunohistochemistry and a graph showing the ratio of AMD2-positive cells.

FIGS. 3A, 3B and 3C illustrate the results of confirming the expression of AMD2 protein in the thyroid carcinoma tissues of thyroid cancer patients with normal body weight (body mass index <25) (A), thyroid cancer patients with obesity (body mass index ≥25) (B), and recurrent thyroid cancer patients with obesity (C) by immunohistochemistry.

In the figures, reference numbers refer to the same or equivalent parts of the present invention throughout the several figures of the drawing.

### DETAILED DESCRIPTION

Hereinafter, exemplary embodiments of the present invention will be described in detail with reference to the accompanying drawings.

In order to accomplish the object of the present invention, the present invention provides a composition for diagnosis or prognosis prediction of thyroid cancer, the composition including an agent for measuring the expression level of mRNA of ADM2 (adrenomedullin 2) gene or its protein.

ADM2 (adrenomedullin 2) gene information is registered in the National Center for Biotechnology Information (NCBI) (NC_000022.11), but the relationship between ADM2 gene and thyroid cancer has not been known at all.

The ADM2 gene of the present invention may preferably include the nucleotide sequence represented by SEQ ID NO: 1, but is not limited thereto. Further, homologs of the nucleotide sequences are included within the scope of the present invention. In particular, the gene includes a nucleotide sequence having a sequence homology of 70% or more, preferably 80% or more, more preferably 90% or more, and most preferably 95% or more, respectively, with the nucleotide sequence represented by SEQ ID NO: 1. “% of sequence homology” to polynucleotides is identified by comparing two optimally aligned sequences by the comparison region, and a portion of the polynucleotide sequence in the comparison region may include addition or deletion (i.e., gap) compared with the reference sequence (without addition or deletion) for the optimal alignment of the two sequences.

In the present invention, the term “diagnosis” is to identify the presence or characteristic of a pathological condition. For the purposes of the present invention, the diagnosis is to ascertain whether or not thyroid cancer has developed.

In the present invention, the term “prognosis” means an increase or decrease in thyroid cancer severity. Prognosis in cancer patients generally refers to metastasis or survival time within a period of time after the cancer onset or surgery. The prognosis prediction suggests the direction of future treatment, in particular, the chemotherapy of the thyroid cancer patients. It can be interpreted as a kind of all the act that predicts the progress after the treatment by comprehensively considering the physiological or environmental condition of the patient after treatment.

Accordingly, for the purpose of the present invention, the prognosis prediction means predicting whether the disease is warned and completely cured after the thyroid cancer treatment, thereby predicting the disease-free survival rate or the survival rate of the thyroid cancer patient. For example, the prediction of “good prognosis” implies that the disease-free survival rate or survival rate of the patient after the thyroid cancer treatment is high and the possibility of treating the thyroid cancer patient is high, and the prediction of “poor prognosis” implies that the disease-free survival rate or survival rate of the patient after the thyroid cancer treatment is low so that there is a high probability of recurrence from thyroid cancer patients and death due to thyroid cancer. The disease-free survival rate means the possibility that the patient can survive without recurrence of cancer after the thyroid cancer treatment, and the survival rate means the possibility that the patient can survive regardless of the recurrence of cancer after the thyroid cancer treatment.

In the present invention, the term “mRNA expression level measurement” is a process for confirming the presence and expression level of the mRNA of the thyroid cancer marker gene (ADM2) in a biological sample for the diagnosis or prognosis prediction of thyroid cancer. To this end, the analysis method includes reverse transcriptase polymerase chain reaction (RT-PCR), competitive RT-PCR, quantified real-time PCR, real-time quantitative RT-PCR, RNase protection assay (RPA), Northern blotting, DNA chip, and the like, but is not limited thereto.

The agent for measuring the mRNA expression level of the ADM2 gene in the present invention may preferably include, but not limited to, a primer, a probe, or an antisense oligonucleotide. The primer, probe, or antisense oligonucleotide may be designed using methods, programs or tools known to those skilled in the art by reference to the nucleotide sequence of the ADM2 gene (the accession number NM_001253845.1).

In the present invention, the term “primer” means a short nucleic acid sequence having a free 3′ hydroxyl group, which is able to form a base pair with a complementary template, and functions as a starting point for amplifying the template strands. The primer can initiate DNA synthesis in the presence of a reagent for polymerization in a suitable buffer solution, at a suitable temperature (i.e., DNA polymerase, or reverse transcriptase) and four different nucleoside triphosphates. In the present invention, PCR amplification can be performed using a sense and antisense primer capable of binding to the base sequence of the ADM2 gene to diagnose or predict the prognosis of thyroid cancer by the production of the desired product. PCR conditions and length of sense and antisense primers can be modified on the basis of the methods known in the art.

In the present invention, the term “probe” means a fragment of nucleic acid such as RNA or DNA, which is several to hundreds of bases capable of specifically binding to mRNA, and is labeled to identify the presence of specific mRNA. The probe can be prepared in the form of oligonucleotide probe, single-stranded DNA probe, double-stranded DNA probe, RNA probe, or the like. In the present invention, hybridization is performed using a probe complementary to the ADM2 polynucleotide, and then thyroid cancer can be diagnosed, or its prognosis can be predicted by the hybridization result. Selection of suitable probe and hybridization conditions can be modified on the basis of the methods known in the art.

In the present invention, the term “measuring the expression level of protein” is a process for confirming the presence and expression level of a protein expressed in a thyroid cancer marker gene (ADM2) from a biological sample for the diagnosis or prognosis prediction of thyroid cancer and may be performed by generally identifying the protein amount. Analysis methods for this may include, but not limited to, western blot, enzyme-linked immunosorbent assay (ELISA), radioimmunoassay (RIA), radioimmunodiffusion, Ouchterlony immunodiffusion, rocket immunoelectrophoresis, immunohistochemistry, immunoprecipitation assay, complement fixation assay, fluorescence activated cell sorting (FACS), protein chips and the like.

In the present invention, the agent for measuring the expression level of the protein is not limited thereto, but may preferably be an antibody or an aptamer that specifically binds to the ADM2 protein.

In the present invention, the term “antibody” is a term known in the art and refers to a specific protein molecule that indicates an antigenic region. With respect to the objects of the present invention, the antibody refers to an antibody that specifically binds to ADM2 protein, the marker of the present invention. The full length or a part of the ADM2 gene is cloned into an expression vector according to a conventional method so as to obtain a protein encoded by the full length or a part of the ADM2 gene and the resulting protein is used as an immunogen (antigen) so that the antibody can be prepared by the conventional method. There is no limitation in the form of the antibody of the present invention, and a polyclonal antibody, a monoclonal antibody, or a functional fragment thereof having antigen-binding property is also included, and all immunoglobulin antibodies are included. Furthermore, the antibody of the present invention also includes special antibodies, such as a humanized antibody. The functional fragment of the antibody molecule refers to a fragment having at least an antigen-binding function and includes Fab, F(ab′) 2, F(ab′)2, ScFv, and the like.

In the present invention, the term “aptamer” refers to a nucleic acid capable of strongly binding specifically to a specific molecule while maintaining a stable tertiary structure. It is compared with antibodies because of its specific binding function and has been evaluated as an alternative technology for antibodies.

The present invention provides a kit for diagnosis or prognosis prediction of thyroid cancer, the kit including the composition as described above.

The kit of the present invention may diagnose thyroid cancer or predict the prognosis of thyroid cancer by identifying the detection of the mRNA of the ADM2 gene or the protein expressed therefrom. The kit for diagnosis or prognosis prediction of thyroid cancer of the present invention includes an agent (for example, a primer or a probe) for detecting the expression level of the ADM2 gene or an agent (for example, an antibody or a aptamer) capable of specifically detecting a protein as well as one or more other component compositions, solutions or devices suitable for the analysis methods.

In the present invention, the kit to assess the mRNA expression level of ADM2 gene may be a kit that includes essential elements required for performing RT-PCR. An RT-PCR kit may include test tubes or other suitable containers, reaction buffers (varying in pH and magnesium concentrations), deoxynucleotides (dNTPs), enzymes such as Taq-polymerase and reverse transcriptase, DNase, RNase inhibitor, DEPC water, and sterile water, in addition to each pair of primers specific to the marker gene. It may include a pair of primers specific to the gene used as a quantitative control.

The kit of the present invention may also be a kit for diagnosis or prognosis, including essential elements required for performing a DNA chip. The DNA chip may include a base plate, onto which cDNAs corresponding to genes or fragments thereof are attached as a probe and reagents, preparations, enzymes, and the like for producing a fluorescent-labeled probe. Further, the base plate may also include a quantitative control gene and cDNA corresponding to fragments thereof.

When the kit of the present invention may be a kit for measuring the expression level of ADM2 protein, it may include a matrix, a suitable buffer solution, a coloring enzyme, or a secondary antibody labeled with a fluorescent substance, a coloring substrate or the like for the immunological detection of antibody. As for the matrix, a nitrocellulose membrane, a 96 well plate made of polyvinyl resin, a 96 well plate made of polystyrene resin, and a sliding glass may be used. As for the coloring enzyme, peroxidase and alkaline phosphatase may be used. As for the fluorescent substance, fluorescein isothiocyanate (FITC) and rhodamine B isothiocyanate (RITC) may be used. As for the coloring substrate solution, ABTS (2,2′-azino-bis-(3-ethylbenzothiazoline-6-sulfonic acid)), OPD (o-phenylenediamine), or TMB (tetramethyl benzidine) may be used.

The present invention provides a method for providing information for the diagnosis of thyroid cancer, the method including:

(1) measuring the expression level of mRNA of the ADM2 gene or protein expressed from the gene from a biological sample isolated from a suspected thyroid cancer patient;

(2) comparing the expression level thereof with that of a normal control sample; and

(3) determining the thyroid cancer if the expression level thereof is over that of the normal control sample.

In the method for providing information for diagnosis of thyroid cancer of the present invention, the measurement of the expression level of mRNA or protein expressed from the gene from a biological sample may be performed by isolating mRNA of a gene or protein thereof from the biological sample, and the isolation of mRNA of the gene or protein thereof may be carried out using the method known in the art.

In the present invention, the term “biological sample” includes, but is not limited to, samples such as tissues, cells, whole blood, serum, plasma, tissue autopsy samples (brain, skin, lymph node, spinal cord, etc.), saliva, sputum, cerebrospinal fluid or urine. However, the biological sample may be used with or without manipulation.

In the method for providing information for diagnosis of thyroid cancer according to the present invention, when the measured expression level of mRNA of the ADM2 gene or protein expressed from the gene from a biological sample isolated from a suspected thyroid cancer patient is over that of the normal control sample, the suspected thyroid cancer patient can be diagnosed as a thyroid cancer patient.

The present invention provides a method for providing information for prognosis prediction of thyroid cancer, the method including:

(1) measuring the expression level of mRNA of the ADM2 gene or protein expressed from the gene from a biological sample isolated from an obese or recurrent thyroid cancer patient;

(2) comparing the expression level thereof with that of a normal body weight thyroid cancer patient sample; and

(3) determining that the risk of thyroid cancer exacerbation is high if the expression level thereof is over that of the normal body weight thyroid cancer patient sample.

The method for providing information for prognosis prediction of thyroid cancer of the present invention may measure the expression level of mRNA of the ADM2 gene or protein expressed from the gene from a biological sample isolated from an obese thyroid cancer patient and then compare the expression level thereof with that of a normal body weight thyroid cancer patient sample, thereby providing information on whether the prognosis of thyroid cancer patient is good or poor. Specifically, when the expression level of the ADM2 gene or protein of an obese thyroid cancer patient sample is over that of the normal body weight thyroid cancer patient sample, it is determined the risk of thyroid cancer exacerbation is high. Further, the method of the present invention may predict the risk of thyroid cancer exacerbation by identifying that the expression level of mRNA of the ADM2 gene or protein expressed from the gene from an obese and recurrent thyroid cancer patient sample is significantly increased compared with that from an obese thyroid cancer patient.

The analysis method for measuring the mRNA level and the analysis method for measuring the protein level are as described above. The comparison between the expression level of the marker gene or protein in normal body weight thyroid cancer patients and the expression level of the marker gene or protein in obese or recurrent thyroid cancer patients may be shown as absolute (e.g., μg/ml) or relative (e.g., the relative intensity of signal) differences.

The present invention provides a method of screening a therapeutic agent for thyroid cancer by measuring the expression level of ADM2 gene or protein encoded by the gene after administering the candidate substance expected to be capable of curing the thyroid cancer.

Specifically, the therapeutic agents may be screened by comparing a change (increase or decrease) of the ADM2 gene or the ADM2 protein under the presence or absence of a candidate substance for curing the thyroid cancer. Preferably an agent that indirectly or directly reduces the expression level of the ADM2 gene or the ADM2 protein can be selected as a therapeutic agent for thyroid cancer. In other words, the expression level of the ADM2 gene or ADM2 protein of the present invention in thyroid cancer cells may be measured in the absence of a candidate substance for curing thyroid cancer, and the expression level of the ADM2 gene or ADM2 protein of the present invention may be measured in the presence of a candidate substance for curing thyroid cancer so that the measured levels may be compared. The substance that reduces the expression level of the ADM2 gene or ADM2 protein of the present invention in the presence of a candidate substance for curing thyroid cancer compared that in the absence of a candidate substance for curing thyroid cancer may be predicted as a therapeutic agent for thyroid cancer.

Hereinafter, the present invention is described in more detail with reference to Examples. However, these Examples are for illustrative purposes only, and the present invention is not intended to be limited by these Examples.

Materials and Methods

1. RNA Sequencing

In the animal model of C57BL/6 mouse with thyroid carcinoma in which the LSL-BRAF mutation is specifically expressed in thyroglobulin, the thyroid tissue of a normal chow diet group or high fat diet (containing 10 times the fat content of normal chow diet) group and the thyroid tissue of control (normal animal model without LSL-BRAF mutation) were stored in Trizol. Then, RNA was extracted using Qiagen's RNA assay kit, and the BAM file was received from the Macrogen company for sequencing. Tuxedo protocol was used to select genes with significant differences in RNA sequencing. The analysis was performed twice: the first analysis was performed by dividing the genes into control and normal diet animal model with thyroid cancer, and second analysis was performed by dividing the genes into normal diet animal model with thyroid cancer and high-fat diet animal model with thyroid cancer.

2. Sample Preparation

2-1. Sample Preparation of Animal Model with Thyroid Cancer

LSL-Braf^{V600E} X TPO-Cre mice previously used in other studies is an animal model in which mutations of LSL-Braf^{V600E} is induced when TPO-Cre is expressed at 14.5 days of the embryonic day, resulting in the induction of thyroid cancer from the developmental stage of mouse thyroid. Thus, it can be inconsistent with the timing of human thyroid cancer. The human thyroid cancer occurs in the form of a thyroid nodule in the normal thyroid parenchyma constituting normal thyroid follicular structure, whereas the existing thyroid-specific carcinoma animal model using TPO-Cre is a maligned model because the follicular structure is destroyed in the entire thyroid so that it is not suitable to reflect the physiology of the patient.

The thyroid cancer animal model used in the present invention was a model in which Tg-Cre/ERT2 mouse (provided by Prof. Jukka Kero of the University of Turku, Finland, genesis, 2014, 52, 333 to 340) and LSL-Braf^{V600E} mouse (provided by Prof. Shioko Kimura of the National Institutes of Health, PNAS, 2011, 108(4), 1615 to 1620) were cross-bred and they were injected with Tamoxifen to express Tg, thereby inducing the mutation of Braf^{V600E}. In order to evaluate whether the 8-week-old thyroid cancer model, which can be considered to have normal thyroid development, was suitable as a carcinoma animal model after adult development, sixteen males were treated with LSL-Braf^{V600E} X Tg-Cre/ERT2 with Tamoxifen for one week to induce mutation of Tg-specific Braf^{V600E} to produce sixteen animal models with thyroid cancer. Eight 8-week-old males were injected with vehicle (saline) to prepare the control group. From the 10th week to the 22nd week, the control and eight thyroid cancer animal models were fed normal diet, and the remaining eight thyroid cancer animal models were fed high fat diet (Casein 265 g/kg, L-Cystine 4 g/kg, Maltodextrin 160 g/kg, Sucrose 90 g/kg, Lard 310 g/kg, Soybean Oil 30 g/kg, Cellulose 65.5 g/kg, Mineral Mix 48 g/kg, Calcium Phosphate 3.4 g/kg, Vitamin Mix 21 g/kg, Choline Bitartrate 3 g/kg and Blue Food Color 0.1 g/kg; ENVIGO, TD.06414) for about 12 weeks. At 22nd weeks of age, mice were sacrificed, and morphological changes of the thyroid gland were observed by immunohistochemical staining (IHC). Histological characteristic was analyzed on eight individuals, and RNA sequencing was performed on nine individuals, including three individuals per group.

2-2. Sample Preparation of Patient with Thyroid Cancer

In order to develop a gene marker for diagnosis and prognosis prediction of thyroid cancer, subjects were collected, and the expression levels of ADM2 thereof were analyzed using their thyroid cancer tissue samples. Of the subjects who were collected, the experiment group was selected based on the criteria shown in Table 1 below. A total of 30 normal body weight thyroid cancer patients, 40 obese thyroid cancer patients, and 10 obese patients with recurrent thyroid cancer were finally selected.

TABLE 1
Thyroid cancer patient classification

| | |
|---|---|
| Normal body weight | Person who has no hypertension, diabetes, |
| patient group | hyperlipidemia, heart disease, stroke |
| among thyroid cancer | (cognitive, non-cognitive). |
| patients | Person who does not take blood coagulant. |
| | Person with a BMI of 19 or more and 25 or less. |
| Obese patient group | Person who has no hypertension, diabetes, |
| among thyroid cancer | hyperlipidemia, heart disease, stroke |
| patients | (cognitive, non-cognitive). |
| | Person who does not take blood coagulant. |
| | Person with a BMI of 25 or more. |
| Recurrent patient group | Person who has no hypertension, diabetes, |
| among obese thyroid | hyperlipidemia, heart disease, stroke |
| cancer patients | (cognitive, non-cognitive). |
| | Person who does not take blood coagulant. |
| | Person with a BMI of 25 or more. |
| | Person who was evaluated to have recurrence by |
| | the ultrasonography of the thyroid for evaluating |
| | the recurrence of thyroid cancer and who was |
| | diagnosed as recurrence of imaging |
| | and biochemistry by confirming the increase of |
| | serum thyroglobulin |

The present invention was conducted with the approval of the Institutional Review Board of Chungnam National University. Clinical questionnaire data collection and human body collection were conducted after obtaining the written consent of the subjects.

3. Immunohistochemical Staining (IHC)

Slides with H & E staining (Hematoxylin and eosin stain) for all cases with good storage and good fixation among 80 cases of patients diagnosed as thyroid cancer were reviewed. Tissues were fixed in formalin, and then the tissues embedded in paraffin were microtome-sliced to 5 μm thickness. The sliced tissues were fixed on slides. They were incubated in a constant temperature oven at 60° C. for 1 hour. Then, they were deparaffinized in a conventional manner, and then treated with different concentrations of alcohol and washed with distilled water. In order to inhibit the activity of endogenous peroxidase, they were treated with 3% aqueous hydrogen peroxide (H₂O₂) for 20 minutes. Then, they were washed with Tris buffer solution (Tris 3.025 mg, 1M NaCl 40 g, 1M HCl 22 ml in H₂O 5 L, pH 7.4). For antigen recovery, the pressure cooker was filled with citrate buffer solution (sodium citrate 14.7 g, 1M HCl 27 ml in H₂O 5 L, pH 6.0) and was heated. The slides were placed at the start of the boiling, and after the pressure reached the maximum (130 kPa), it was boiled for 2 more minutes, and immersed in cold water to lower the pressure. The slides were taken out and placed in Tris buffer solution. To prevent nonspecific reactions, the blocking antibody was reacted at room temperature for 10 minutes, and the primary antibody was reacted. Anti-adrenomedullin 2 antibody (SC-140883, Santa Cruz Biotechnology, USA) was used as the primary antibody. After the primary antibody reaction, they were washed with Tris buffer solution, and LSAB+kit (DAKO, Japan) was used to react with the secondary antibody at room temperature for 30 minutes. After completion of the reaction, they were washed with water to complete the color reaction. Then, they were stained with Harris hematoxylin, dehydrated with alcohol, sealed, and observed with a microscope.

### Example 1. Analysis of Role of ADM2 in Animal Model with Thyroid Cancer

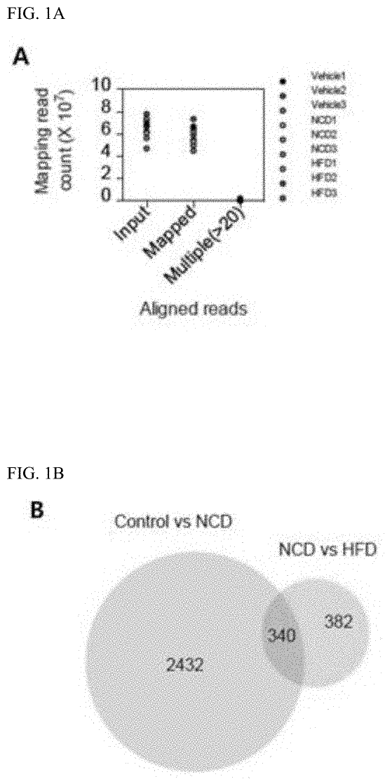

In order to analyze the role of ADM2 in animal models with thyroid cancer, expression levels of ADM2 in animal model tissues were identified. As a result, it was observed that the high-fat diet group (HFD) showed the increase in the size of thyroid cancer and the number of proliferating cells compared with the normal chow diet (NCD) in the animal model with the thyroid cancer. In order to identify the mechanism, RNA sequencing was performed. As a result, about 2,732 genes were differentiated in the cancer-free control and NCD animal models, and about 722 genes were statistically differentiated in the NCD animal model and the HFD animal model. Among these, ADM2 (adrenomedullin 2) gene was found to be a gene which differs in all of the control, NCD and HFD (See FIG. 1).

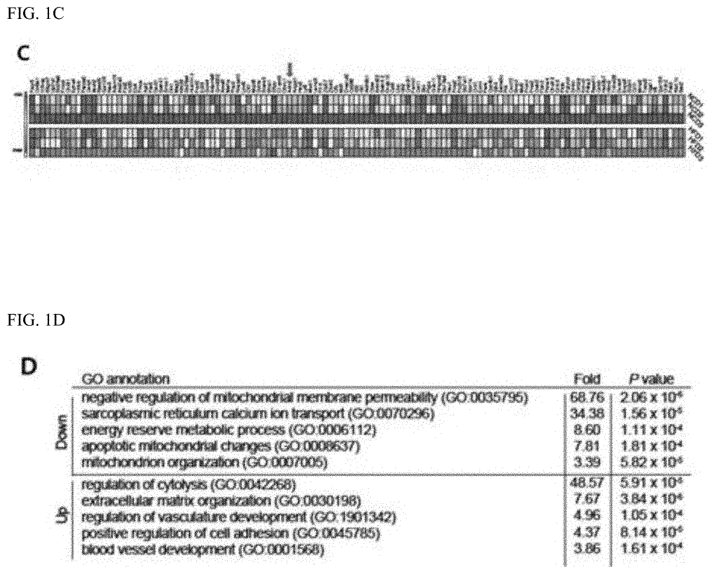

ADM2 protein expression in the animal model with thyroid cancer was confirmed, and the results indicated that the expression of ADM2 protein in the HFD animal model was significantly increased as compared with the NCD animal model (See FIG. 2).

### Example 2. Analysis of Role of ADM2 in Patient with Thyroid Cancer

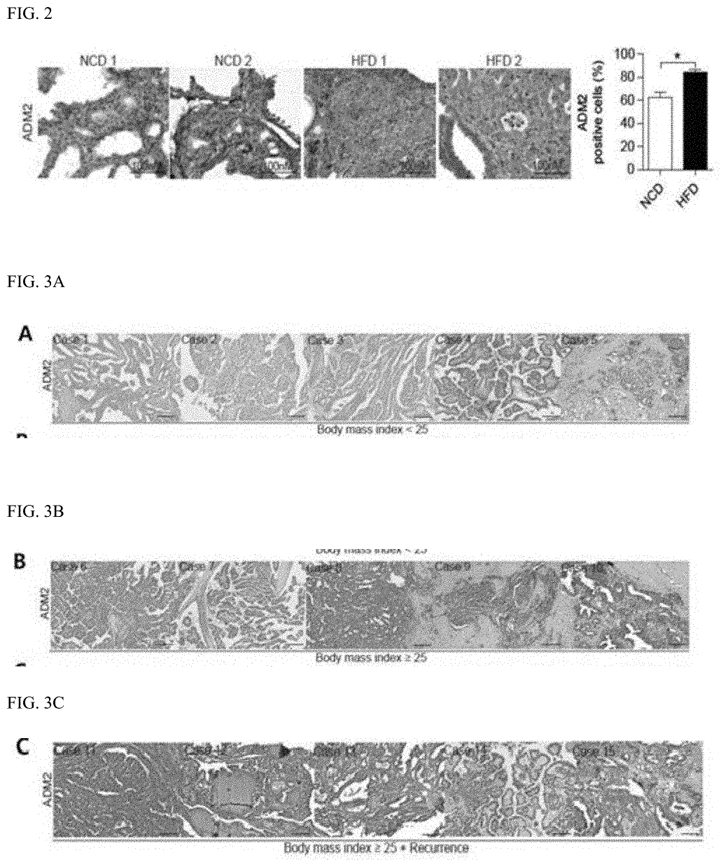

In order to analyze the role of ADM2 in patients with thyroid cancer, the expression level of ADM2 of thyroid cancer patient sample was identified. The results indicated that compared to those of normal thyroid cancer (FIG. 3A), the ADM2 expression was increased in tissues of thyroid cancer (FIG. 3B) of obese patients with thyroid cancer, whose clinical features such as lymph node metastasis and recurrence of thyroid cancer were worse, and the ADM2 expression was further increased in tissues of thyroid cancer (FIG. 3C) of obese patients with recurrent thyroid cancer compared to those of obese patients with thyroid cancer.

The above results suggest that the expression level of ADM2 gene can be used as a therapeutic composition for solving intractable thyroid cancer because it can predict the risk of recurrence after treatment of thyroid cancer as well as the relationship between obesity and exacerbation of thyroid cancer.

As described above, the preferred exemplary embodiments have been described, but the scope of the present invention is not limited thereto. Various modifications and improvements by those skilled in the art using the basic concept of the present invention defined in the following claims are also within the scope of the present invention.

As described above, the exemplary embodiments have been described and illustrated in the drawings and the specification. The exemplary embodiments were chosen and described in order to explain certain principles of the invention and their practical application, to thereby enable others skilled in the art to make and utilize various exemplary embodiments of the present invention, as well as various alternatives and modifications thereof. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A composition for diagnosis or prognosis prediction of thyroid cancer, the composition comprising an agent for measuring an expression level of mRNA of ADM2 (adrenomedullin 2) gene or its protein.

2. The composition of claim 1, wherein the agent for measuring the level of mRNA of the gene includes a primer, a probe, or an antisense oligonucleotide that specifically binds to the gene.

3. The composition of claim 1, wherein the agent for measuring the level of the protein includes an antibody or an aptamer specific for the protein.

4. A kit for diagnosis or prognosis prediction of thyroid cancer, the kit comprising the composition according to claim 1.

5. The kit of claim 4, wherein the kit is an RT-PCR kit, a DNA chip kit or a protein chip kit.

6. A method for providing information for diagnosis of thyroid cancer, the method comprising:
(1) measuring an expression level of mRNA of ADM2 gene or protein expressed from the gene from a biological sample isolated from a suspected thyroid cancer patient;
(2) comparing the expression level thereof with that of a normal control sample; and
(3) determining that thyroid cancer has developed if the expression level thereof is over that of the normal control sample.

7. A method for providing information for prognosis prediction of thyroid cancer, the method comprising:
(1) measuring an expression level of mRNA of ADM2 gene or protein expressed from the gene from a biological sample isolated from an obese or recurrent thyroid cancer patient;
(2) comparing the expression level thereof with that of a normal body weight thyroid cancer patient sample; and
(3) determining that the risk of the thyroid cancer exacerbation is high if the expression level thereof is over that of the normal body weight thyroid cancer patient sample.

8. A method of screening a therapeutic agent for thyroid cancer, the method comprising measuring an expression level of ADM2 gene or protein encoded by the gene after administering a candidate substance expected to be capable of curing the thyroid cancer.
